(19) United States
(12) Patent Application Publication
(10) Pub. No.: US 2014/0276826 A1
(43) Pub. Date: Sep. 18, 2014

(54) TARGETING GUIDE FOR AN INTRAMEDULLARY NAIL

(71) Applicant: Biomet Trauma, LLC, Warsaw, IN (US)

(72) Inventors: Anthony J. Metzinger, Winona Lake, IN (US); Kevin T. Stone, Winona Lake, IN (US)

(21) Appl. No.: 14/215,622

(22) Filed: Mar. 17, 2014

Related U.S. Application Data
(60) Provisional application No. 61/789,934, filed on Mar. 15, 2013.

(51) Int. Cl. A61B 17/72; A61B 17/74; A61B 17/88

(52) U.S. Cl. CPC A61B 17/72; A61B 17/8897; A61B 17/744
USPC 606/62

(57) ABSTRACT

A targeting instrument system for positioning an intramedullary implant. The system comprises a multi-use targeting guide arm, an intramedullary implant, and a single-use nose segment configured to maintain a locking alignment between the intramedullary implant and the targeting guide arm during the placement of the intramedullary implant into a subject. The system may be provided as a sterile kit, and the intramedullary implant may be preassembled to the nose segment. Alternatively, system may include a sterile kit comprising a single-use targeting guide arm, and an intramedullary implant, wherein the intramedullary implant is preassembled to the single-use targeting guide arm.

### CROSS-REFERENCE TO RELATED APPLICATIONS

This application claims the benefit of U.S. Provisional Application No. 61/789,934, filed on Mar. 15, 2013. The entire disclosure of the above application is incorporated herein by reference in its entirety.

### INTRODUCTION

The present technology generally relates to surgical instruments and procedures, and more particularly, to targeting guide devices, their associated instrumentation, and procedures for the repair of fractured bones.

Hip fracture nail (HFN) systems, also broadly referred to as reconstruction nail systems, are available for surgically treating a wide range of proximal femoral fracture indications. HFN systems typically include an intramedullary nail sized and shaped for surgical implantation into a intramedullary canal of the fractured, proximal femur. The proximal portion of the nail has a smooth, transverse bore that retains a lag screw or the like having a distal end that anchors into the femoral head of the femur, such that the construct holds the femoral neck and the diaphysis (shaft) of the femur at a fixed angle with respect to each other, while allowing “sliding compression” of the fractured, proximal femur to promote proper healing. Typically this neck/shaft angle is in the range of about 125 to about 130 degrees. The proximal portion of the nail may also include another transverse, smooth bore that retains an anti-rotation screw alongside and proximal to the lag screw. Manufacturers typically provide HFN systems with both long and short versions of the nail and in various sizes to accommodate patient anatomy variations.

Surgeons usually implant the hip fracture nail and screws with the aid of an x-ray radioscope (fluoroscope) in order to verify proper reduction of the fracture and to properly position the nail and screws in the femur. It may be important to insert the distal end of the lag and anti-rotation screws into the central portion of the femoral neck and head so as not to weaken the construct or to break out through the articulation surface of the femoral head. Accordingly, manufacturers provide special instrumentation for implanting the HFN system. Such instrumentation typically includes a targeting guide device, or jig, that attaches to the end of the intramedullary nail.

The target jig, which may include the aid of radioscopic visualization, provides a handle for holding and positioning the nail into the femur. The target jig also includes target holes aligned with the lag screw and anti-rotation screw holes in the nail, to aid the surgeon in drilling the pilot holes into the femoral neck and head to receive the lag and anti-rotation screws. Portions of the target jig may be radiolucent in order to radioscopically visualize the nail, while other portions of the target jig may be radiopaque in order to provide visual references for aligning and positioning the nail inside the femur so that the axis of the lag screw passes approximately through the center of the femoral neck and head.

It is usually necessary for the surgeon to take several radioscopic images in the medial-lateral and anterior-posterior directions in order to reduce the fracture and to properly position the nail in the femur. This is primarily because it is often difficult for the surgeon to discern if the radioscopic view is optimal for directing a guide wire through the femoral neck and to the proper depth in the femoral head. The guide wire is needed for guiding a cannulated drill to create a pilot hole for the lag screw. Clearly, each radioscopic image increases exposure of the surgeon, staff, and patient to radiation and adds to the surgical procedure time and costs. What is needed, therefore, is improved instrumentation to aid the surgeon in properly implanting a hip fracture nail into the femur of a patient. There is even a further need to provide tools and methods in a relatively economical manner that includes disposable/single-use components in addition to, or in place of, reusable components.

### SUMMARY

This section provides a general summary of the disclosure, and is not a comprehensive disclosure of its full scope or all of its features.

The present teachings provide a targeting instrument system for positioning an intramedullary implant. The system comprises a targeting guide arm, an intramedullary implant, and a single-use nose segment. The single-use nose segment is configured to maintain a locking alignment between the intramedullary implant and the targeting guide arm during the placement of the intramedullary implant into a subject.

The present teachings also disclose a disposable surgical kit for a targeting instrument system. The kit comprises a single-use targeting guide arm and an intramedullary implant. The intramedullary implant is preassembled to the single-use targeting guide arm and stored together in a sealed, sterile container.

Still further, the present teachings provide a sterile surgical kit for a targeting instrument system. The sterile surgical kit comprises a multi-use targeting guide arm, a single-use nose segment, and an intramedullary implant. The intramedullary implant is coupled to the single-use nose segment and preassembled to the multi-use targeting guide arm and stored together in a sealed, sterile container.

Further areas of applicability of the present teachings will become apparent from the description provided hereinafter. It should be understood that the description and specific examples are intended for purposes of illustration only and are not intended to limit the scope of the present teachings.

### BRIEF DESCRIPTION OF THE DRAWINGS

The present teachings will become more fully understood from the detailed description and the accompanying drawings, wherein:

FIG. 1 is an anterior-posterior view of a hip fracture nail (HFN) prosthesis assembly, which includes an intramedullary (IM) nail and a lag screw defining a lag screw axis, implanted into a proximal femur of a subject according to various aspects of the present disclosure;

FIG. 2 is a side plan view of a targeting instrument system according to one aspect of the present teachings, shown with a single-use targeting guide arm in locking alignment with the IM nail of FIG. 1;

FIG. 3 is a side plan view of a targeting instrument system according to another aspect of the present teachings, shown with a multi-use targeting guide arm removably coupled to a single-use nose segment in locking alignment with the IM nail of FIG. 1; and

FIG. 4 is a detailed perspective view of a shaft of an exemplary nose segment in connecting alignment with the IM nail.

It should be noted that the figures set forth herein are intended to exemplify the general characteristics of materials, methods, and devices among those of the present technology, for the purpose of the description of certain aspects. These figures may not precisely reflect the characteristics of any given aspect, and are not necessarily intended to define or limit specific embodiments within the scope of this technology. Further, certain aspects may incorporate features from a combination of figures.

### DESCRIPTION OF VARIOUS EMBODIMENTS

The following description of technology is merely exemplary in nature of the subject matter, manufacture, and use of one or more inventions, and is not intended to limit the scope, application, or uses of any specific invention claimed in this application or in such other applications as may be filed claiming priority to this application, or patents issuing therefrom.

The present technology generally relates to instruments useful for the positioning of medical implant components and methods for improving the procedures for implanting medical devices and bone fracture repairs. As used herein, the term “implant” may be used to refer to an entire implant, or a portion thereof; portions may be as large or as small as necessary to accommodate the specific need. For example, an implant made in accordance with the present disclosure, generally including a nail and lag screw as shown in FIG. 1, may constitute the entire implant, or it may be used with one or more pieces or components that together form a final implant or implant assembly. The present disclosure encompasses a wide variety of therapeutic and cosmetic applications, for human and/or other animal subjects, and the specific materials, devices, and instruments used should be biomedically acceptable. As used herein, such a “biomedically acceptable” or “biocompatible” material or component is one that is suitable for use with humans and/or animals without undue adverse side effects (such as toxicity, irritation, and allergic response) commensurate with a reasonable benefit risk/ratio.

The terminology used herein is for the purpose of describing particular example embodiments only and is not intended to be limiting. In this disclosure, the terms “anterior,” “posterior,” “lateral,” and “medial” generally refer to the front, back, outside, and midline of a surgical patient, respectively, although these terms are also used in reference to instruments and/or devices. It should also be noted that the term “user” may refer to a surgeon or any one of a number of individuals who assist the surgeon during a bone fracture repair procedure. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Targeting instrument systems such as targeting guide devices, or jigs, are often used for guiding drills, guide wires, and screws into or thru an intramedullary or extramedullary implant device. One such device can be found in pending U.S. patent application Ser. No. 13/796,138, filed Mar. 12, 2013, incorporated herein by reference in its entirety. Targeting guide devices are often used as multi-use devices. For example, they may be delivered to an operating room, along with various other instruments, after being cleaned and sterilized in a standard process of washing and autoclaving as is known in the art. This may create extra steps for cleaning and sterilization within a traditional autoclave process.

The present technology envisions manufacturing and packaging the targeting instrument systems with the guide device either completely disposable, or at least partially disposable. For example, targeting instrument systems with certain reusable, or multi-use components and certain disposable, or single-use components. In certain aspects, the present technology provides sterile, sealed surgical kits including various components that can be individually selected and combined together for the intended purpose. In one aspect, if the entire targeting instrument system is disposable, i.e., intended only for a single use, it could be packaged and pre-assembled as a targeting guide arm coupled to an implant, saving timely assembly steps in an operating room. If the targeting guide device includes a separate nose segment mechanism that is meant to be disposable, it could be packaged and preassembled to the implant for placement onto a multi-use targeting guide arm, also reducing the amount of assembly time in the operating room. Certain kits may include a plurality of single-use nose segments, each having a different size or shape. Certain kits may additionally or alternatively include a plurality of intramedullary implants, each having a different size or shape.

According to the present technology, the targeting instrument systems may be distributed as a disposable or reusable surgical kit in a sterilized container to an operating room. As such, the targeting device that is attached to the implant is intended for immediate use in the operating room without needing additional cleaning or sterilization. In certain aspects, the kit is re-sterilizable by a steam autoclave once the sealed container has been opened. The surgical kits may include various other components that may be used in the implant procedure, such as the sterile guide wires and sterile drill guides.

It should be understood that while most of the discussion herein pertains to IM nail targeting guide devices, the present technology can be incorporated with and similarly be used for other surgical tools and/or devices that may need to attach targeting mechanisms to order to place screws or guide wires.

In an effort to provide related context to the present technology, FIG. 1 illustrates an anterior-posterior view of a hip fracture nail prosthesis assembly 20 (or HFN prosthesis 20) implanted into a proximal portion of a femur 22 having a femoral head 24 with a center 26, a femoral neck 28, a greater trochanter 30, an intramedullary canal 32, and a femoral diaphysis 34. Shown as a basic assembly, the HFN prosthesis 20 includes at least an intramedullary implant nail 36 (or IM nail 36) and a lag screw 38. The IM nail 36 includes a proximal shaft portion 40 defining a proximal shaft axis 42 and a distal shaft portion 44 defining a distal shaft axis 46 that may be slightly inclined relative to proximal shaft axis 42 to conform to the shape of femur 22. The lag screw 38 defines a lag screw axis 48 and includes a proximal barrel portion 50 and a distal threaded portion 52. A transverse bore in the proximal portion 40 of the IM nail 36 slidingly retains the barrel portion 50 of the lag screw 38. The lag screw axis 48 and distal shaft portion axis 46 define a femoral neck-diaphysis angle indicated by the letter A, which corresponds approximately to the angle formed between the femoral neck 28 and the femur diaphysis 34. For most patients, this angle can approximately be in the range of about 125-135 degrees.

The distal threaded portion 52 of the lag screw 38 is designed for threadable engagement into the bone in the center 26 of the femoral head 24. However, it should be noted that there are different, but functionally equivalent, devices for anchoring into the femoral neck 28 and femoral head 24, including “blade” types of lagging devices for use with femoral nails, and reference to the distal threaded portion 52 of the lag screw 38 is not intended to be limiting.

The HFN prosthesis 20 may optionally include an end cap 54 and a retaining screw (hidden) inside of the internally threaded, proximal portion 40 of the IM nail 36 for retaining the lag screw 38. All of the components of the HFN prosthesis 20 may be formed from any one or more of a number of biocompatible, radiopaque materials, including a titanium alloy and stainless steel, as is well known in the art.

FIG. 1 also shows a first plane 56 that is orthogonal to a second plane 58. The intersection of the first plane 56 and the second plane 58 coincides with the lag screw axis 48. A first line of sight 60 is contained in the first plane 56. A second line of sight 62 is contained in the second plane 58. The first line of sight 60 and the second line of sight 62 may generally correspond to the optimal set-up directions of a radioscopic imaging device when positioning the IM nail 36 in the femur 22 in order to direct a targeting guide wire along the lag screw axis 48 and through the center 26 of the femoral head 24. Once properly inserted into femoral neck 28 and femoral head 24, a targeting guide wire may be used to safely guide a cannulated drill to create a pilot hole for the lag screw 38, as is well known in the art.

The actual direction of the first line of sight 60 within the first plane 56 with respect to the lag screw axis 48 may vary depending on the skill level of the user. Optimally, however, the first line of sight 60 is approximately perpendicular to the lag screw axis 48 and within the first plane 56. This may be especially significant when inserting a guide wire into the femoral head 24 to the desired depth to prevent penetration of the guide wire tip through the articulation surface of the femoral head 24. Similarly, the actual direction of the second line of sight 62 within the second plane 58 with respect to the distal shaft axis 46 may vary, depending again on the skill level of the user. As shown, the optimal direction of the first line of sight 60 is approximately parallel to the lag screw axis 48 and within the second plane 58. As will be described, particular features of the devices disclosed herein may assist the user in radioscopically visualizing the IM nail 36 along the first and second lines of sight 60, 62 in order to properly set the insertion depth and the version angle of the IM nail 36 in the femur 22.

The HFN prosthesis 20 may also include an anti-rotation screw (not shown) as noted previously. The present disclosure, however, is directed primarily to disposable devices, kits, and procedures associated with the proper positioning of implants, such as the IM nail 36 and the lag screw 38 into the femur 22, since the positioning of the anti-rotation screw into the femur 22 is predetermined by the prior positioning of the lag screw 38 into the femur 22. The HFN prosthesis 20 may also include additional screws and other internal components not described herein. Additional description of an exemplary device that is generally similar to the HFN prosthesis 20 and that includes an anti-rotation screw may be found in U.S. Pat. App. No. 2006/0106386 entitled “Orthopaedic Screw and Method” filed by E. Reber, et al., on Jun. 28, 2005, incorporated by reference in its entirety.

FIG. 2 is a side plan view of a first targeting instrument system 63 including a single-use, disposable targeting guide arm 64 (or target jig 64) shown in connecting alignment with the IM nail 36. FIG. 3 is a side plan view of a second targeting instrument system 65 including a two-component targeting guide device having a multi-use targeting guide arm 66, i.e., intended to be reusable, coupled to a disposable or single-use nose segment 68 shown coupled to the multi-use targeting guide arm 66 and in connecting alignment with the IM nail 36. The targeting instrument systems 63, 65 may be substantially U-shaped when fully assembled, and include a curved or arcuate-shaped handle portion 67 having a superior end 68 and an inferior end 70. Both the disposable targeting guide arm 64 and the multi-use targeting guide arm 66 may be formed from a radiopaque, biocompatible polymeric material such as carbon-filled polyether ether ketone (PEEK).

As shown in FIG. 2, the disposable targeting guide arm 64 can be a monolithic component having a connection end 74 coupled to the IM nail 36. In certain aspects, a connecting bolt may also be provided, configured for coupling the single-use targeting guide arm 64 to the IM nail 36. In other aspects, the single-use targeting guide arm 64 may be configured to detachably couple to the intramedullary implant via at least one of a rotary quick-connect fitting, a bayonet fitting, a threaded coupling, a press-fit coupling, and a snap-fit coupling, the respective details of which are commonly known in the art. Alternatively, the system shown in FIG. 2 may additionally include a separate single-use or multi-use nose segment coupled between the IM nail 36 and the single-use targeting guide arm 64 configured to maintain a locking alignment between the IM nail 36 and the single-use targeting guide arm 64.

As shown in FIG. 3, the nose segment 68 removably connects to the superior end 72 of the multi-use targeting guide arm 66. The removable connection 76 between the nose segment 68 and the multi-use targeting guide arm 66 may be selected from at least one of a rotary quick-connect fitting, a bayonet fitting, a threaded coupling, a press-fit coupling, and a snap-fit coupling. As shown, the nose segment 68 includes an arm connecting portion 78 and a shaft portion 80 having an end 82 that extends to define a nose axis 81 that is coaxial with the proximal shaft axis 42 (FIG. 1) of the IM nail 36 when the IM nail 36 is attached to the targeting guide device 65. The nose segment 68 can be provided with an alternate shape, if desired. As shown, the connecting portion 78 of the nose segment 68 may extend substantially orthogonal to the shaft portion 80, which is generally parallel with the nose axis 81. The nose segment 68 may be formed from a biocompatible, radiopaque polymeric material. If desired, the nose segment 68 could also be formed from a stronger material, such as a titanium alloy or a stainless steel.

The handles 67 of the targeting instrument systems 63, 65 may include a plurality of wire holes defining various respective target axes (not shown for simplicity). For example, the inferior end 70 of the handles 67 may include a lag screw target hole defining a lag screw target axis as is known in the art. When the IM nail 36 is coupled to the targeting instrument systems 63, 65, the lag screw hole of the IM nail 36 is aligned with the lag screw target axis. A clamp element 84 may be assembled to the inferior end 70 of the handle 66. Once attached to the targeting instrument systems 63, 65, the IM nail 36 can be manipulated into the femur 22 such that the lag target axis passes approximately through the center 26 of the femoral head 24 prior to drilling a pilot hole for the lag screw 38.

FIG. 4 illustrates a detailed perspective view showing one aspect of the shaft 80 of the nose segment 68 in connecting alignment with the IM nail 36. As shown, the connection end 96 of the nose shaft 80 may optionally include a plurality of spaced apart spring fingers 98 forming a circle centered on the nose component axis 81. The fingers 98 may be sized and configured to deflect and then to grip the inside of the IM nail 36 when inserted to a desired depth. The axial force required to removably connect an aligned nose segment 68 to an IM nail 36 may be predetermined by adjustment of the overall spring rate of the individual spring fingers 98. As is well known, various mechanical attachment designs are possible to provide a holding force at a desired insertion distance. In various aspects, the nose segment 68 of the targeting guide device 65 and the IM nail 36 include respective interlocking features preventing rotation of the IM nail 36 with respect to the targeting guide device 65. As shown, the connection end 96 may include a plurality of radially spaced apart flutes 102, 104. The IM nail 36 may include a plurality of recesses 106 having appropriate widths to receive the flutes 102, 104. It should be understood that the specific design, configuration and number of flutes, recesses, protrusions, and the like can be varied as known in the art such that they effectively lock the spatial orientation, alignment, and relative movement between the targeting guide device 64, IM nail 36, and/or the holding device 65.

Additional specific descriptions of other instrument components in relation to hip fracture nail systems can also be found in U.S. Pat. No. 8,257,354 and pending application Ser. No. 13/633,913 (published on Jan. 31, 2013 as U.S. Pub. No. 2013/0030444), the entire specifications of which are incorporated herein by reference.

The foregoing discussion discloses and describes merely exemplary arrangements of the present teachings. The embodiments described herein are not intended to be limiting in describing the full scope of implant devices and methods of the present technology. Equivalent changes, modifications and variations of embodiments, materials, components, and methods can be made within the scope of the present technology, with substantially similar results. Furthermore, the mixing and matching of features, elements, and/or functions between various embodiments is expressly contemplated herein, so that one of ordinary skill in the art would appreciate from this disclosure that features, elements and/or functions of one embodiment may be incorporated into another embodiment as appropriate, unless described otherwise above. Moreover, many modifications may be made to adapt a particular situation or material to the present teachings without departing from the essential scope thereof. One skilled in the art will readily recognize from such discussion, and from the accompanying drawings and claims, that various changes, modifications and variations can be made therein without departing from the spirit and scope of the present teachings.

What is claimed is:

1. A targeting instrument system for positioning an intramedullary implant, the system comprising:
a targeting guide arm;
an intramedullary implant; and
a single-use nose segment configured to maintain a locking alignment between the intramedullary implant and the targeting guide arm during the placement of the intramedullary implant into a subject.

2. The targeting instrument system of claim 1 provided as a sterile kit in a sterile container, wherein the single-use nose segment is preassembled to the intramedullary implant.

3. The targeting instrument system of claim 1 provided as a sterile kit in a sterile container, comprising a plurality of single-use nose segments, each single-use nose segment having a different size or shape.

4. The targeting instrument system of claim 1 provided as a sterile kit in a sterile container, comprising a plurality of intramedullary implants, each intramedullary implant having a different size or shape.

5. The targeting instrument system of claim 1, wherein at least one or both of the intramedullary implant and a connection end of the single-use nose segment comprise respective interlocking features preventing rotation of the intramedullary implant with respect to the targeting guide arm.

6. The targeting instrument system of claim 1, wherein the single-use nose segment is configured to detachably couple to the targeting guide arm via at least one of a rotary quick-connect fitting, a bayonet fitting, a threaded coupling, a press-fit coupling, and a snap-fit coupling.

7. The targeting instrument system of claim 1, wherein the single-use nose segment is configured to detachably couple to the intramedullary implant via at least one of a rotary quick-connect fitting, a bayonet fitting, a threaded coupling, a press-fit coupling, and a snap-fit coupling.

8. The targeting instrument system of claim 1, wherein the single-use nose segment comprises a radiopaque polymeric material.

9. The targeting instrument system of claim 1, further comprising a connecting bolt configured for coupling the single-use nose segment to the intramedullary implant.

10. A disposable surgical kit for a targeting instrument system, the surgical kit comprising:
a single-use targeting guide arm; and
an intramedullary implant,
wherein the intramedullary implant is preassembled to the single-use targeting guide arm and stored together in a sealed, sterile container.

11. The surgical kit according to claim 10, wherein the single-use targeting guide arm is configured to detachably couple to the intramedullary implant via at least one of a rotary quick-connect fitting, a bayonet fitting, a threaded coupling, a press-fit coupling, and a snap-fit coupling.

12. The surgical kit according to claim 10, further comprising a single-use nose segment coupled between the intramedullary implant and the single-use targeting guide arm and configured to maintain a locking alignment between the intramedullary implant and the single-use targeting guide arm.

13. The surgical kit according to claim 10, further comprising a connecting bolt configured for coupling the single-use targeting guide arm to the intramedullary implant.

14. The surgical kit according to claim 10, wherein the single-use targeting guide arm comprises a radiopaque polymeric material.

15. The surgical kit according to claim 10, further comprising at least one of a sterile wire guide and sterile drill guide.

16. The surgical kit according to claim 10, wherein the surgical kit is re-sterilizable by a steam autoclave once the sealed container has been opened.

17. A sterile surgical kit for a targeting instrument system, the sterile surgical kit comprising:
a multi-use targeting guide arm;
a single-use nose segment; and
an intramedullary implant,
wherein the intramedullary implant is coupled to the single-use nose segment and preassembled to the multi-use targeting guide arm and stored together in a sealed, sterile container.

18. The sterile surgical kit according to claim 17, wherein the single-use nose segment is configured to detachably couple to the multi-use targeting guide arm via at least one of a rotary quick-connect fitting, a bayonet fitting, a threaded coupling, a press-fit coupling, and a snap-fit coupling.

19. The sterile surgical kit according to claim 17, further comprising a connecting bolt configured for coupling the single-use nose segment to the intramedullary implant.

20. The sterile surgical kit according to claim 17, further comprising at least one of a sterile wire guide and sterile drill guide.
